(19) United States
(12) Patent Application Publication
(10) Pub. No.: US 2011/0077642 A1
(43) Pub. Date: Mar. 31, 2011

(54) PLASMA APPLICATORS FOR PLASMA-SURGICAL METHODS

(76) Inventor: Günter Farin, Tübingen (DE)

(21) Appl. No.: 12/809,490

(22) PCT Filed: Dec. 17, 2008
(86) PCT No.: PCT/EP2008/010785

§ 371 Date: Nov. 23, 2010

(30) Foreign Application Priority Data
Dec. 20, 2007 (DE) 10 2007 061 482.0
Jan. 17, 2008 (DE) 10 2008 004 843.7

(51) Int. Cl. A61B 18/12

(52) U.S. Cl. 606/34

(57) ABSTRACT

Electrosurgical instruments that transmit electrical energy from an electrosurgical generator via an electrode and a current path of ionized gas into biological tissue. In order to obtained a defined, low treatment depth in the target tissue, the electrosurgical instrument contains a resistive element with a predetermined impedance between the distal end of the connection line and the electrode, installed in such a way that treatment current is limited after ionizing of the gas.

### FIELD OF THE INVENTION

The disclosed embodiments relate to plasma applicators for the use of plasma surgery in open surgery or in rigid endoscopy and plasma probes for the use of plasma-surgical methods in flexible endoscopy.

### BACKGROUND

Generally in plasma surgery (shown schematically in FIG. 6), ionized and consequently electrically conductive gas (plasma), i.e. argon plasma, is used to direct high-frequency electrical AC current (HF current, I_{HF}) onto or into biological tissue (target tissue, A, B, C, D) in order to generate medically beneficial thermal effects on or in the target tissue, in particular devitalization (D), coagulation (C), desiccation (B) and/or shrinkage (A) without damaging collateral tissue (G) any more than is unavoidable and tolerable. The ionization of the gas between an ionization electrode (E) and the target tissue occurs with a sufficiently high electrical field strength (Fₑ) corresponding to the function Fₑ=U_{HF}:d. Ionization of argon at atmospheric pressure requires approximately 500 V/mm.

Plasma-surgical methods and plasma applicators are not new. One such plasma-surgical method, fulguration or spray coagulation, has been used for more than 50 years for thermal haemostasis in surgical operations. Fulguration or spray coagulation employs primarily oxygen and nitrogen plasmas which are generated in air. These plasmas are chemically reactive and give rise to carbonization effects, pyrolysis effects and consequently vaporization of tissue and smoke on the surface of the tissue. Such side effects disrupt and can even prevent the use of fulguration or spray coagulation, especially for endoscopic operations.

U.S. Pat. No. 4,060,088 describes limiting the above-described side effects of fulguration or spray coagulation by replacing the air between the active electrode and tissue being treated with a chemically inert or noble gas, such as helium or argon and mixtures thereof. Nowadays, argon is mainly used due to its relatively low cost. This method, known as argon plasma coagulation (APC), has been in use almost 20 years. An early example of a clinically applicable device for APC is described in U.S. Pat. No. 4,781,175, but that device is only for use in open surgery and rigid endoscopy for thermal haemostasis.

Use of APC for flexible endoscopy was first described in 1994 by Farin and Grund (G. Farin, K. E. Grund: Technology of Argon Plasma Coagulation with Particular Regard to Endoscopic Applications. Endoscopic Surgery and Allied Technologies, No. 1 Volume 2, 1994: 71-77; and K. E. Grund, D. Storek, G. Farin: Endoscopic Argon Plasma Coagulation (APC): First Clinical Experiences in Flexible Endoscopy. Endoscopic Surgery and Allied Technologies, No. 1 Volume 2, 1994: 42-46). The range of applications of APC in flexible endoscopy was later described by Grund, Zindel and Farin (K. E. Grund, C. Zindel, G. Farin: Argon Plasma Coagulation in Flexible Endoscopy: Evaluation of a New Therapeutic after 1606 Uses. Deutsche Medical Wochenschrift 122, 1977: 432-438).

APC is not used only for the coagulation of biological tissue. APC is also used for thermal devitalization of pathological tissue and desiccation and shrinking of blood vessels and their collateral tissue for purposes of haemostasis. This use of APC is becoming increasingly important for the thermal devitalization of relatively thin layers of tissue such as the mucosa of the gastrointestinal tracts or tracheobronchial system. APC is also becoming increasingly important for the thermal sterilization of the surface of tissue during transmural operations, for example in transgastral operations, in order to avoid the dissemination of germs from the stomach into the abdominal cavity. Because such uses are not adequately defined by the term argon plasma coagulation (APC), and the subject matter to be discussed in this application is not solely restricted to the coagulation of biological tissue only or the use of argon gas, we will use the broader and more comprehensive term “plasma surgery.”

The broad range of applications for plasma surgery places different requirements on the properties of the devices used, in particular the reproducibility of the intended thermal effects on or in target tissue and the avoidance of the side effects common to air-based fulguration or spray coagulation described above. This is especially the case when plasma surgery is used on or in thin-walled hollow organs in the gastrointestinal tract or the tracheobronchial system (As described in G. Farin, K. E. Grund: Principles of Electrosurgery, Laser, and Argon Plasma Coagulation with Particular Regard to Colonoscopy. In: Colonoscopy; Principles and Practice, Edited by J. D. Waye, D. K. Rex and C. B. Williams, Blackwell Publishing 2003: 393-409).

In fact, the range of different plasma applicators for medical applications, in particular plasma surgery and endoscopically controlled interventions, is very broad. As far as access to the respective target organ or target tissue, known plasma applicators may be differentiated between those used for open surgery, those used for rigid endoscopy and those used for flexible endoscopy. The basic structure and function of such plasma applicators can be seen in G. Farin and K. E. Grund: Technology of Argon Plasma Coagulation with Particular Regard to Endoscopic Applications, Endoscopic Surgery and Allied Technologies, Thieme Verlag, Stuttgart, No. 1, Volume 2, 1994: 71-77.

An arrangement for endoscopic plasma surgery is shown in FIG. 7. Generally, endoscopic plasma surgery requires a surgical high-frequency (HF) generator 1, which is connected to a neutral electrode 2 and a surgical instrument or probe 10 (or its discharge electrode, which is not shown). The probe 10 is inserted in one of the working channels 6 of an endoscope 5. Argon (or another noble gas) is fed from a noble gas source 7 to a lumen of the probe 10. The neutral electrode 2 is placed in contact with the patient's biological tissue 3. In this way, the operator can treat target tissue 4 with argon plasma.

HF generators available for plasma surgery may be differentiated with respect to their internal resistance. HF generators with a high internal resistance are in particular suitable for treating superficial lesions, with which a low penetration depth of the thermal effects is expedient. HF generators with a low internal resistance are in particular suitable for treating massive lesions, where a high penetration depth of the thermal effects is expedient. DE 19839826 describes an HF generator in which the internal resistance is adjustable between high and low, but such HF generators are not yet available.

For treatment of superficial lesions, low penetration depth of thermal effects is desired and often necessary. As such, it can be problematic to employ low internal resistance HF generators because penetration depth is primarily controlled by duration of application, i.e., by the operator. Since the rate of penetration of the thermal effects at the start of the plasma application with low internal resistance HF generators is relatively quick and becomes progressively slower over time until the maximum achievable penetration depth is achieved, the achievement of a low and uniform penetration depth of the thermal effects with surface lesions is extremely difficult, if not impossible. Although the rate of penetration of the thermal effects can be influenced by varying the power or HF current flowing through the plasma, the pulse modulation can interfere with video signals from video endoscopes and give rise to neuromuscular stimuli, the latter in particular at modulation frequencies of less than 1 kHz. With known HF generators, this is done by considering the high HF voltage required to ionize the gas by pulse modulation. A further problem with the use of HF generators with low internal resistance to treat superficial lesions is the very high temperature of the plasma due to the high HF voltage required for the ionization and of the low electrical resistance of the plasma path, which results in a very high HF current density in the plasma path and may result in a high enough temperature to cause carbonization and pyrolysis effects.

However, HF generators with high internal resistance are essentially unusable for endoscopic operations or interventions because of stray capacitance between the active HF lines and neutral HF line. As such, the transmission of high HF voltage between the ionization electrode and target tissue required for the ionization of the gas from the HF generator to the ionization electrode is inadequate or worse, impossible. This is particularly well documented with respect to flexible endoscopy.

EP 1 148 770 A2 describes a plasma applicator where none of the neutral electrodes normally provided are used. The HF generator provided there is supposedly a resonance transmitter, wherein the HF current flows as a “dielectric displacement current from the surface of the patient to earth”. This is supposed to induce scabbing with a surgical cold plasma jet apparatus that can take place without carbonization or combustion products caused by oxygen inclusion.

### SUMMARY

The disclosed embodiments include an electrosurgical instrument having a resistive element in a hand piece of the instrument in series connection with the electric circuit. This resistive element has a fixed position, and remains in the hand piece even when the electrode is connected separately. Such an electrosurgical instrument is both inexpensive and safe, and adequately addresses the problems noted above with respect to controlling penetration depth of thermal effects.

Another embodiment comprises an electrosurgical instrument for the transmission of electrical energy from an electrosurgical HF generator via a connection line and an electrode connected to a distal end thereof and further via a current path of ionized gas into a biological target tissue. Disposed between the distal end of the connection line and the electrode is a resistive element having predetermined impedance. The element is selected in such a way that ensures limitation of treatment current after gas ionization.

An important point is that because the entire arrangement including the generator and all leads is considered to be the “entire generator,” the internal resistance of the entire generator can be determined by the resistive element. The resistive element can thus be selected to meet requirements for a desired penetration depth, and it is therefore possible to provide different instruments for different penetration depths. Obviously, treatment time is still significant, but there is significant added control during the critical phase (the brief moment following the “ignition” of the arc during which a high current flow is possible).

The resistive element can be an ohmic resistance which ensures the desired current limitation. However, the resistive element is preferably capacitive in nature, i.e., a capacitive element with necessary dielectric strength. When a capacitive element is used, it forms a high-pass filter so that low-frequency portions of the current are dampened. This in turn leads to a significant reduction in interference to video systems (such as those commonly used in endoscopes) and avoidance of neuromuscular stimuli that can occur with lower frequency current.

In the case of sufficiently large plasma applicators, the capacitive element can be implemented by commercially available components, for example, a ceramic capacitor capable of resisting high voltage. But in rigid or flexible endoscopes (as described in connection with FIG. 7), such as, for example, argon-plasma coagulation probes (APC probes) such as those described in DE 19820240 or DE 10129699 or EP 1397082, the instrument channels are quite narrow, often times with an external diameter of only 2 to 3.5 mm. As such, the capacitive element itself has to be specially developed (to fit in the small space) or the distal end of the APC probe must be altered.

Since, as mentioned above, APC probes can be used with other gases or gas mixtures, for other thermal effects and optionally also in other specialist fields, in the following, plasma applicators such as those described herein will be called general plasma probes (“P probes”).

In a preferred embodiment, the resistive element comprises a segment of the connection line and/or the electrode, i.e. segments of these components are used either for the formation of an ohmic resistance or (optionally) for the formation of a capacitance. The resistive element can be formed from parallel-guided or twisted or coaxially-arranged segments of the connection line, which are electrically insulated from each other and/or a supply line to the electrode and/or the electrode itself. In this case, it should be ensured that no inductances form, for instance, because of the use of bifilar line arrangements.

Generally, the resistive element has a capacitance of 10 pF to approximately 1,000 pF. These are capacitance ranges that result in currents at the frequencies usually used in high-frequency surgery, thus ensuring the desired (relatively low) penetration depth.

The dielectric used to create the capacitive element should have the highest possible dielectric constant. Plastics can be used, but ceramic materials are generally preferred. The material used can be rigid ceramic material or even (if greater flexibility is required) powdery ceramic material.

All the above features can also be used in electrosurgical arrangements in which no “protective gas” is used, though it is preferable to work with a protective gas (in particular argon). Generally, the electrode is in or close to a tube, a hose or a probe and is positioned in such a way that the gas to be ionized can be supplied to a chamber between the electrode and the target tissue.

### BRIEF DESCRIPTION OF THE DRAWINGS

The following describes an exemplary embodiment of the invention in more detail with reference to the attached diagrams:

FIG. 1 shows a schematic representation of an embodiment of the disclosed electrosurgical instruments,

FIGS. 2-5 show schematic representations of resistance elements embodied as capacitive elements and the associated electrodes,

FIG. 6 shows the representation described in the introduction to explain the processes during argon plasma coagulation and

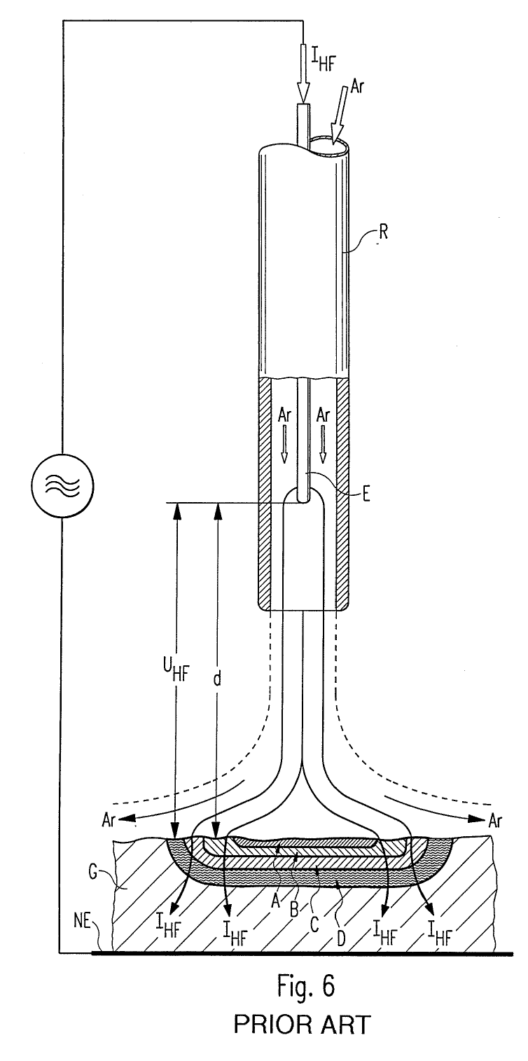

FIG. 7 shows an overall arrangement for the endoscopic treatment of tissue by means of APC.

### DETAILED DESCRIPTION

In the following description, the same reference numerals denote the same objects or objects having similar functions.

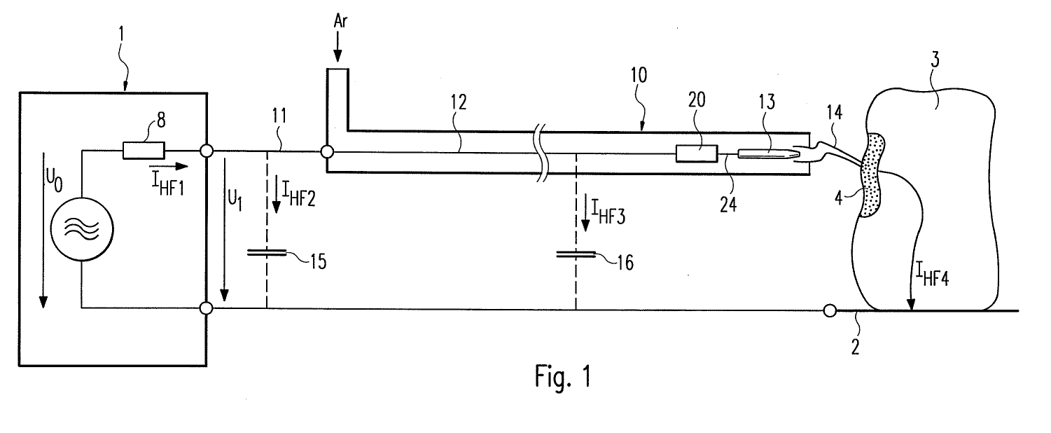

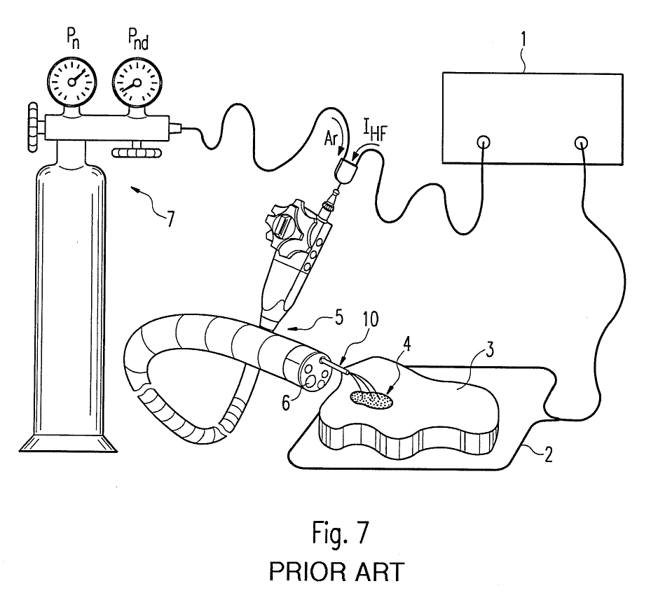

FIG. 1 shows a schematised arrangement of an embodiment of the electrosurgical instrument disclosed herein that corresponds to the plasma surgical instrument shown in FIG. 7. The endoscope shown in FIG. 7 is not seen in this arrangement. However, as mentioned above, arrangements of this kind can also be used for open surgery which does not require an endoscope.

As shown FIG. 1, a high-frequency generator is provided having a voltage source with a voltage U₀ and an internal resistance 8 with a resistance value Rᵢ. Thus, where there is an output current I_{HF1} at the output from the generator 1, there is a voltage U₁

U₁=U₀−Rᵢ*I_{HF1}

at the output terminals of the generator 1. The generator is connected via a supply line 11 to a probe supply line 12 arranged within a hose of the probe 10. The probe line 12 is connected at its distal end via a resistive element 20 and an electrode supply line 24 to an electrode 13. Argon gas is conducted through the hose of the probe 10 so a chamber between the distal end of the probe 10 and the biological tissue 3 is filled with argon gas and the air normally found there is forced out. When the voltage between the tip of the electrode 13 and the biological tissue 3 is high enough, the gas (argon) in this chamber between the electrode 13 and the biological tissue 3 is ionized, and an arc 14 forms. Then, a current I_{HF4} flows through the target area 4 and surrounding biological tissue 3 to the neutral electrode 2.

The supply line 11 is usually a monopole line. In addition, the neutral electrode 2 is kept at the surrounding potential (as is an optionally provided endoscope) so that there is a relatively high stray capacitance 15 between the supply line 11 and a stray capacitance 16 between the probe line 12 and the surroundings. Currents I_{HF2} or I_{HF3} flow through these stray capacitances 15 and 16. This stray capacitance causes a drop in the voltage (U_{Z}) used between the electrode 13 and the target area 4 for the ignition of plasma before the ignition of an arc 14:

U_{Z}=U₀−R_{I} (I_{HF2}+I_{HF3})

In order to ensure the ignition of the plasma 14 at the greatest possible distance from target area 4, it is advantageous for the value R_{I} of the internal resistance 8 to be low. On the other hand, when arc 14 is ignited it has a very low resistance, and due to the fact that the resistance between the target area 4 and the neutral electrode 2 is also relatively small, there will be a very high current I_{HF4}. As such, only a short time will pass before target area 4 is affected at a relatively deep depth.

Resistive element 20 is thus disposed between the distal end of the (high-loss) line 11, 12 and the electrode 13, so that even with a high ignition voltage available at the electrode 13 following the ignition of the arc 14, a high voltage drop is generated to limit current I_{HF4}. This limiting is made possible by having the resistive element 20 at this position. However, it should be stressed that the resistive element 20 does not have to locally limit the current. The resistor element 20 can in fact extend over the length of the (high-loss) line 11, 12 up to the tip of the electrode 13.

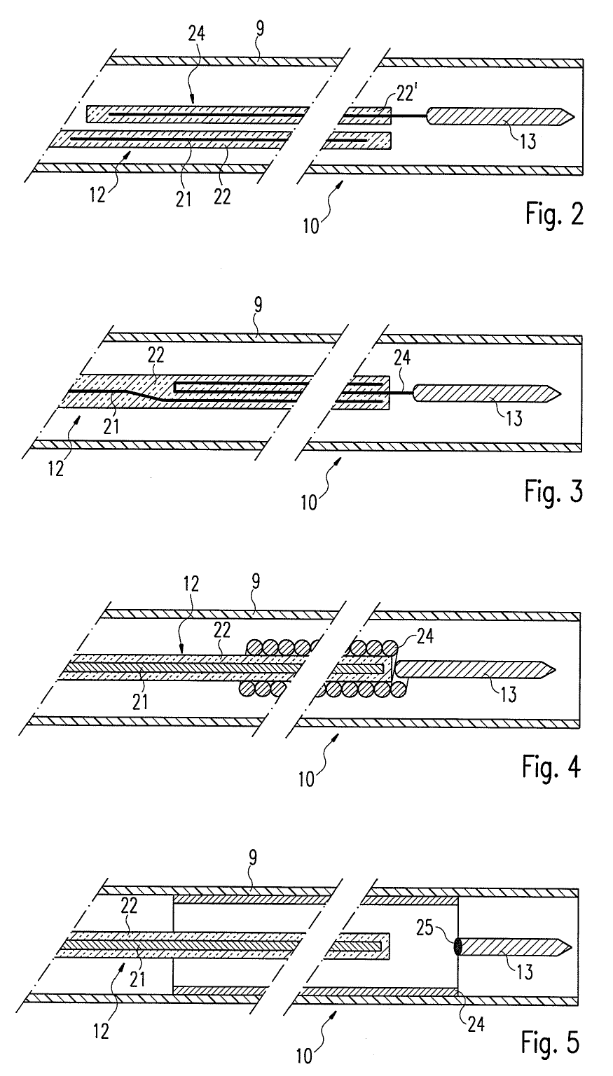

The following describes different embodiments of the resistive element 20 with reference to FIGS. 2-5.

In the embodiment shown in FIG. 2, a distal end of the probe line 12 is shown comprising a probe conducting wire 21 insulated by insulating material 22. Disposed parallel to this distal end of the probe line 12 is the electrode supply line 24, which is connected to the electrode 13 and provided with insulation 22′. Parallel guidance of the two lines 12/24 results in the formation of a capacitance which functions as resistive element 20.

The embodiment shown in FIG. 3 differs from the FIG. 2 embodiment in that both the distal end of the probe conducting wire 21 and the end of the electrode supply line 24 are embedded in a common insulating material 22. In addition, the electrode supply line 24 is bifilar so that any line inductances are compensated. In such a case, ceramic material can be used as insulating material (solid or powder form) to achieve the highest possible capacitance in the smallest space.

In the embodiment shown in FIG. 4, the capacitance is increased by winding the electrode supply line 24 around the end of the probe supply line 12. The electrode supply line 24 can be bifilar here as well to compensate for line inductances.

In the embodiment shown in FIG. 5, the electrode supply line 24 is embodied as a sleeve surrounding the distal end of the probe line 12 and forming a capacitance with said probe line. In this case, the electrode is in an electrically conductive connection via a connecting point 25 with the sleeve-shaped electrode supply line 24. The dimensions can also be similar to those in FIG. 4, so the supply of argon gas no longer flows through the sleeve-shaped electrode supply line 24, but past it into the hose 9 of the probe 10.

No matter the embodiment, it is important to provide suitable insulation so that there is no disruptive discharge between elements formed by lines 12/24. It is also possible to embed the lines 12/24 in a wall of the hose 9 that forms the gas line for the probe 10. The working channel 6 of the endoscope 5 can also be used as a gas line, as described in EP 0954246 A1.

The physical parameters that determine the capacitance between the lines can be found in technical literature. Further, the arrangement and shape of the gas outlet opening can obviously be embodied not only, as shown in the exemplary embodiments, in the axial direction, they can also be arranged differently, such as shown for example in DE 19820240 A1 or DE 10129699 A1.

In sum, limiting the amplitude of the HF current flowing through the plasma, as the above described embodiments do, provides not only control of the penetration depth of thermal effects in target tissue, but several other advantages, including: avoidance of excessively high plasma temperatures and hence avoidance of carbonization or even pyrolysis of the target tissue; avoidance of thermal overloading of the distal end of the plasma probe, i.e., when the plasma comes into direct contact with plastic (such as with the plasma probes of DE 10129699); avoidance of interference with video systems; and avoidance of neuromuscular stimuli, which are both prevented by the capacitive resistance's mitigation of low-frequency currents.

It should be pointed out here that all the above described parts and in particular the details illustrated in the drawings are essential for the disclosed embodiments alone and in combination. Adaptations thereof are the common practice of persons skilled in the art.

1-10. (canceled)

11. An electrosurgical instrument comprising:
a probe for treating a target area of biological tissue with electrical energy from an electrosurgical HF generator, the electrical energy being delivered to the target area via a connection line to an electrode connected to a distal end of the connection line and further via an arc of ionized gas to the target area; and
a resistive element having a predetermined impedance disposed between the distal end of the connection line and the electrode, the resistive element being configured to limit treatment current after gas ionization.

12. The electrosurgical instrument of claim 11, wherein the probe is tubular or hose-shaped.

13. The electrosurgical instrument of claim 11, wherein the probe is housed in the working channel of an endoscope.

14. The electrosurgical instrument of claim 11, wherein the electrical energy is returned to the electrosurgical energy via a neutral electrode.

15. The electrosurgical instrument of claim 11, wherein the resistive element is a capacitance.

16. The electrosurgical instrument of claim 11, wherein the resistive element is a commercially available resistor or capacitor.

17. The electrosurgical instrument of claim 11, wherein the resistive element comprises a segment of the connection line and/or the electrode.

18. The electrosurgical instrument of claim 11, wherein the connection line and a line to the electrode are not physically connected.

19. The electrosurgical instrument of claim 18, wherein the resistive element comprises parallel-guided or twisted or coaxially-arranged segments of the connection line and the line to the electrode and/or the electrode itself.

20. The electrosurgical instrument of claim 19, wherein the segments are arranged bifilarly.

21. The electrosurgical instrument of claim 11, wherein the resistive element has a capacitance of 10 pF to 1,000 pF.

22. The electrosurgical instrument of claim 11, wherein the resistive element comprises a ceramic material as insulation and/or a dielectric.

23. The electrosurgical instrument of claim 22, wherein the ceramic material is a powder.

24. The electrosurgical instrument of claim 11, wherein the electrode is attached in or close to a tube or hose in the probe and the tube or hose is configured to supply gas to be ionized into a chamber between the electrode and target area.

25. A method for treating a target area of biological tissue, the method comprising:
supplying electrical energy to the target area from a probe connected to an electrosurgical HF generator, the electrical energy being supplied via a connection line in the probe to an electrode connected to a distal end of the connection line and further via an arc of ionized gas to the target area; and
limiting treatment current after gas ionization by causing the electrical energy to flow through a resistive element of a predetermined impedance disposed between the distal end of the connection line and the electrode.

26. The method of claim 25, wherein the connection line and a line to the electrode are not physically connected.

27. The method of claim 26, wherein the resistive element comprises parallel-guided or twisted or coaxially-arranged segments of the connection line and the line to the electrode and/or the electrode itself.

28. The method of claim 25, further including returning the electrical energy to the electrosurgical HF generator via a neutral electrode in contact with the biological tissue.

29. The method of claim 25, further including supplying gas to be ionized into a chamber between the electrode and target area via a tube or hose in the probe that the electrode is attached in or close to.
